(19) United States
(12) Patent Application Publication
(10) Pub. No.: US 2023/0086682 A1
(43) Pub. Date: Mar. 23, 2023

(54) METHOD FOR SUPPORTING IMPROVEMENT OF SLEEP AND ELECTROLYTIC HYDROGEN GENERATOR USED FOR SAME

(71) Applicant: Aqua Bank CO.,LTD., Osaka-shi, Osaka (JP)

(72) Inventor: Takashi TAKEHARA, Osaka-shi, Osaka (JP)

(21) Appl. No.: 17/909,225

(22) PCT Filed: Mar. 8, 2021
(86) PCT No.: PCT/JP2021/008922

§ 371 Date: Sep. 2, 2022

(30) Foreign Application Priority Data
Mar. 6, 2020 (JP) 2020-039115

(51) Int. Cl. A61M 15/02; A61M 21/02; C25B 1/04

(52) U.S. Cl. CPC A61M 15/02; A61M 21/02; C25B 1/04

(57) ABSTRACT

Provided are a method for sleep improvement obtained by regular inhalation of air containing high-concentration hydrogen, an electrolytic hydrogen generator and a portable electrolytic hydrogen generator for implementing the method. According to the method, air containing high-concentration hydrogen from a nozzle portion of a portable electrolytic hydrogen generator is inhaled through a mouth or a nose by continuously using the electrolytic hydrogen generator that releases air containing high-concentration hydrogen from the nozzle portion when necessary. The generator enables an operation while being held with one hand, the control substrate controls power supply and stop of power supply from a battery to positive and negative electrodes in response to an operation of the operation means, the control including control for supplying power for a predetermined time in response to an operation of the operation means and stopping power supply after a lapse of the predetermined time.

### TECHNICAL FIELD

The present invention relates to a method for supporting sleep improvement of a user who is aware of sleeplessness and an electrolytic hydrogen generator suitable for implementing the method, in which air containing high-concentration hydrogen is inhaled through a mouth or a nose by continuously using the portable electrolytic hydrogen generator that releases air containing high-concentration hydrogen when necessary.

### BACKGROUND ART

The usefulness of hydrogen has received attention in recent clinical trials on humans, and various studies on hydrogen have been conducted in medical application. Hydrogen is administered into a human body by various methods including intravenous administration, oral administration with an aqueous solution, and gas inhalation (inhalation through a nose and a mouth). Conventionally, clinical trials have been conducted on the administration and eye drops or the like of a hydrogen-containing aqueous solution, but definite clinical trials about gas inhalation have not been provided. The inventors provide various clinical trials and verifications on healthy people, who are not targets of medical application, by using hydrogen inhalation in addition to clinical trials and verifications in medical application. In particular, the inventors provide clinical trials and verifications in which an antistress effect or a cognitive function are improved by a single-dose inhalation or continuous use of hydrogen for young women or healthy elderly people and a dementia risk-factor amount (MCI risk factor) makes a significant change, so that hydrogen inhalation means and a dedicated device thereof are provided to obtain a significant effect (Patent Literature, see a patent of aqua hydrogen).

Furthermore, the inventors and the applicant have received a large amount of feedback suggesting improvements in sleep by hydrogen inhalation from subjects and ordinary users in the process of the clinical trials and verifications. For example, a subject easily gets to sleep and is not waken at night. Actually, the effect of hydrogen inhalation on sleep has not been specifically verified by the inventors or other third parties, and the inventors only made an assumption but have not provided social information.

The problem of sleep has become a social problem in recent years and is broadly divided into the problem of “sleeping habits” caused by the problem of a body clock, for example, insufficient sleep or shift work and the problem of “sleep disorders,” such as sleep apnea and insomnia. It is understood that poor sleep quality may increase the risk of lifestyle-related diseases and cause worsening of symptoms. Moreover, the inventors found that working women tend to be aware of sleeplessness among subjects in clinical trials and verifications.

In such circumstances, as described above, the inventors assumed that hydrogen inhalation preferably affects sleep. The inventors conducted clinical trials and verifications on subjects to prove the influence of continuous use of hydrogen inhalation and devised a method and a design that are most suitable for adequate sleep by continuously using hydrogen inhalation. The subjects are, in particular, working women who tend to be aware of sleeplessness.

### CITATION LIST

### Patent Literatures

Patent Literature 1: Japanese Patent Laid-Open No. 2004-41949

Patent Literature 2: Japanese Patent Application No. 2014-019640

Patent Literature 3: WO 2018/151107

### SUMMARY OF INVENTION

### Technical Problem

The present invention has been devised in view of such circumstances. According to findings about the significant effect of hydrogen inhalation on sleep, an object of the present invention is to provide a method for supporting sleep improvement, by which an advantageous effect on sleep is obtained by regular inhalation of air containing high-concentration hydrogen through the mouths or noses of users including healthy people who are aware of sleeplessness, and simultaneously provide an electrolytic hydrogen generator suitable for implementing the method.

### Solution to Problem

In order to solve the problem, as described below, the present invention provides a method for supporting sleep improvement of a user who is aware of sleeplessness and a portable electrolytic hydrogen generator used for supporting sleep improvement of the user.

First, provided as a first present invention is a method for supporting sleep improvement of a user who is aware of sleeplessness, in which air containing high-concentration hydrogen is continuously inhaled from a nozzle portion of a portable electrolytic hydrogen generator for a predetermined time or longer each time through a mouth or a nose multiple times per day, by continuously using the electrolytic hydrogen generator that releases air containing high-concentration hydrogen from the nozzle portion when necessary.

Specifically, in the method for supporting sleep improvement, it is preferable that air containing high-concentration hydrogen is inhaled from the nozzle portion of the electrolytic hydrogen generator through a mouth or a nose for about five minutes or longer per dose and at least five times per day, and the inhalation is continuously used for about two weeks or longer.

As in the method for supporting sleep improvement according to the first present invention, significant improvement in sleep quality can be supported by intake through regular inhalation through a mouth or a nose under natural breathing while continuously using air containing high-concentration hydrogen. As will be described later, this time, as a recommendation example, continuous inhalation was conducted using a portable electrolytic hydrogen generator for about five minutes per day, five times per day by users (particularly working women) who are aware of sleeplessness, so that an improvement in awareness of sleeplessness, a significant increase in sleep efficiency, significant reductions in sleep-onset time and out-of-bed time, and a significant decrease in stress were confirmed. This proved that at least the continuous hydrogen inhalation using the electrolytic hydrogen generator reduces daily stress and improves sleep quality, and the findings were provided. This assumes to be a significant advantage of the present invention (a second present invention described later).

The method for supporting sleep improvement recommends hydrogen inhalation for about five minutes or longer per dose and at least five times per day and for about two weeks or longer. The recommendation example derives from the confirmation of a significance in a verification test, which will be describe later, and an actual significance confirmed under set (devised) conditions with a lower threshold value preset on the basis of a balance between an amount of hydrogen in blood and the probability of continuous use by a user. The detail will be described later. The lower threshold value of hydrogen inhalation is set at five minutes per dose based on a reperfusion phenomenon and the effects of antioxidation and bloodstream restoration. The setting of hydrogen inhalation to be conducted at least five times per day is based on a time during which a hydrogen concentration in blood is eliminated, the typical habit of taking supplements or the like by users, and practicability from habits specific to an object to support sleep improvement. Thus, the present invention is advantageous in that the method for supporting sleep improvement with high practicability is also provided for healthy people who do not need doctor's guidance.

As second present invention provides a portable electrolytic hydrogen generator used for supporting sleep improvement for a user who is aware of sleeplessness, by continuously using inhalation of air containing high-concentration hydrogen through a mouth or a nose, the electrolytic hydrogen generator including:

a body cover member including a battery, a control substrate for controlling power supply from the battery, and a pair of positive and negative electrodes in which a positive electrode and a negative electrode are energized or shut down by the control substrate;

a transparent or translucent electrolysis tank that is attached to the body cover member, accommodates the inserted pair of positive and negative electrodes, and is capable of storing water;

a mixing part including a passage that fluidly connects a nozzle portion and the electrolysis tank by a dedicated flow path and draws ambient air, the nozzle portion allowing inhalation through a mouth or a nose while being held with one hand; and

operation means enabling an operation of the electrolytic hydrogen generator held with one hand, wherein the control substrate controls power supply and stop of power supply from the battery to the positive and negative electrodes in response to an operation of the operation means, the control including control for supplying power for a predetermined time in response to an operation of the operation means and stopping power supply after a lapse of the predetermined time.

The electrolytic hydrogen generator according to the second present invention is a portable electrolytic hydrogen generator that releases air containing high-concentration hydrogen from the nozzle portion, as a dedicated device used for the method for supporting sleep improvement of a user who is aware of sleeplessness according to the first present invention. As described above, in the first present invention, daily stress is reduced and sleep quality is improved by intake through regular inhalation of air containing high-concentration hydrogen through a mouth or a nose under natural breathing. As a dedicated device that can properly achieve the improvement, characteristic configurations are used in the electrolytic hydrogen generator. First, the electrolytic hydrogen generator is a portable device that allows hydrogen inhalation when necessary. The electrolytic hydrogen generator can be operated while being held with one hand during use, and electrolysis is used for generating hydrogen. As described in the first present invention, in order to obtain a significant effect as support for sleep improvement, hydrogen inhalation for about five minutes or longer and at least five times per day in daily life is proposed. For simple use at any locations, e.g., at home under the continuous use conditions, a certain amount of hydrogen needs to be generated with a small and portable device, proving the necessity for electrolysis that facilitates the control of an amount of generated hydrogen according to an electromotive force with a small space. Since the invention is intended to support improvement in sleep quality usually by being used before bedtime, if a user is requested to perform complicated regular operations in frequent use at bedtime, it may be difficult to expect a proper effect. It is preferable to provide the function of continuing hydrogen emission for a necessary time in response to an operation. Thus, a used control configuration includes a configuration in which at least an operation for generating hydrogen and stopping the generation can be easily performed by single operation means held with a hand during hydrogen inhalation, power is supplied for a predetermined time in response to an operation of the operation means, and power supply is stopped after a lapse of the predetermined time. Furthermore, subjects are healthy people including working women who do not intend to be healthy first, and thus it is significant to keep continuous use without tiredness. In the case of the electrolytic hydrogen generator, the transparent or translucent electrolysis tank allows a user to confirm the generation of bubbles of hydrogen and oxygen in the electrolysis tank at bedtime and easily confirm inhalation of proper gas. This point is also advantageous.

Specifically, in the electrolytic hydrogen generator is configured as follows: the operation means has a single operation button allowing operation by pressing, the control substrate performs control to supply power from the battery to the positive and negative electrodes when the operation button is pressed and a pressing state is kept while a main power supply is turned on, and the control substrate performs control to stop power supply from the battery to the positive and negative electrodes when the pressing state is released, the control substrate performs control to supply power from the battery to the positive and negative electrodes when the operation button is pressed once while a main power supply is turned on, and the control substrate performs control to stop power supply from the battery to the positive and negative electrodes after a lapse of a preset time.

Moreover, the operation means preferably performs control to turn off the main power supply after a lapse of a predetermined time following the stop of power supply from the battery to the positive and negative electrodes.

In the example of the electrolytic hydrogen generator, an operation for turning on/off the main power supply and an operation for energization/shutdown related to hydrogen generation are performed by a pressing operation button. In some configurations, hydrogen is generated by continuously pressing the operation button in an inhalation position while the main power supply is turned on, so that an inhalation time is easily obtained. In other configurations, energization to the positive and negative electrodes by pressing the operation button allows hydrogen generation for a recommended time, so that an inhalation time is secured. Moreover, a control configuration may be used to turn off the main power supply to save power after a lapse of a predetermined time (preferably 20 minutes) following the end of a recommended inhalation time. The configurations were devised for power saving and based on sleep-onset time data, and the detail will be described layer.

The electrolytic hydrogen generator preferably includes an LED that illuminates the electrolysis tank, and

the control substrate preferably energizes the LED when power is supplied from the battery to the positive and negative electrodes.

Moreover, the electrolytic hydrogen generator preferably includes:

a sleep support cartridge that is heated by energization and releases gas containing aromatic components or a supplement with an enhanced sleeping effect,

wherein the control means controls energization or shutdown to the pair of electrodes and/or the sleep support cartridge, and

hydrogen and gas containing aromatic components or a supplement released by energizing the sleep support cartridge are preferably mixed in the mixer and are guided to the nozzle portion for inhalation through a mouth or a nose, the hydrogen being generated by energizing the pair of positive and negative electrodes so as to electrolyze an electrolyte solution or water in the electrolysis tank.

In the case of the electrolytic hydrogen generator, power for heating the sleep support cartridge containing effective components for sleep improvement can be supplied during hydrogen inhalation in addition to continuous inhalation of air containing high-concentration hydrogen as support for sleep improvement, thereby further promoting and supporting an improvement in sleep quality according to the style of a user.

### Advantageous Effect of Invention

The present invention provides a method for supporting sleep improvement, by which an advantageous effect on sleep is obtained by regular inhalation of air containing high-concentration hydrogen through the mouths or noses of users including healthy people who are aware of sleeplessness, and simultaneously provide an electrolytic hydrogen generator suitable for implementing the method.

### BRIEF DESCRIPTION OF DRAWINGS

FIGS. 1A and 1B indicate the analysis results of the PSQI-J total scores of a group A and a group B according to a sleep questionnaire (Pittsburgh Sleep Quality Index).

FIGS. 2A and 2B indicate the analysis results of the total scores of the group A and the group B according to the stress check list, 30 items (SCL30).

FIGS. 3A and 3B indicate the analysis results of physical activities during sleep, FIG. 3A indicates sleep efficiency, and FIG. 3B indicates a sleep-onset time (sleep latency time).

FIGS. 4A, 4B, and 4C indicate the analysis results of physical activities during sleep, FIG. 4A indicates the analysis result of the number of tosses and turns, FIG. 4B indicates the analysis result of an arousal time during sleep, and FIG. 4C indicates the analysis result of out-of-bed time (out-of-bed latency time).

FIG. 5 is a block diagram schematically illustrating an embodiment of an electrolytic hydrogen generator according to the present invention.

FIGS. 6A, 6B, 6C and 6D show a front view, left and right side views, and a plan view of the embodiment of the electrolytic hydrogen generator according to the present invention.

FIG. 7 is a schematic diagram illustrating a state of electrolysis in an electrolysis tank of the electrolytic hydrogen generator according to the present invention.

FIG. 8 schematically illustrates a specific configuration of an electrode.

FIGS. 9A, 9B, and 9C show external view photographs of the embodiment of the electrolytic hydrogen generator according to the present invention.

### DESCRIPTION OF EMBODIMENT

First, an example of a method for supporting sleep improvement according to the present invention (hereinafter simply referred to as “the method for supporting sleep improvement”) and the demonstration result will be described below.

<<Outline of Sleep Evaluation Test by Continuous Use of Hydrogen Inhalation and Analysis Result>>

In a verification test in the method for supporting sleep improvement, hydrogen generated by an electrolytic hydrogen inhaler 1 (“Kencos 4” by Aqua Bank Co., Ltd. is used as a verification of a hydrogen inhaler for health improvement (sample)) is inhaled through a mouth. In a preferred configuration example of the electrolytic hydrogen gas inhaler 1, the amount of generated oxygen-hydrogen mixture gas containing 8 cc of hydrogen and 4 cc of oxygen sums to 12 cc per minute of use by electrolysis of a solution (in an actual inhalation, an environmental atmosphere (atmosphere) is also included). The configuration example will be specifically described later.

In the present verification test, subjects were working women (22 women at ages 30 to 45) who live in the Tokyo area and its vicinity and are aware of sleeplessness (difficulty in falling asleep, arousal during sleep, difficulty in getting up, and light sleep or the like).

As a test method, the subjects were randomly divided into two groups (group A, group B). The subjects of the group A were tested for a total of two weeks: a week with hydrogen inhalation using the electrolytic hydrogen gas inhaler 1 (sample use week) and then were tested for a week without using the electrolytic hydrogen gas inhaler 1 (control week), and the subjects of the group B were tested for two weeks including a week without using the electrolytic hydrogen gas inhaler 1 (control week) and then were tested for a week with hydrogen inhalation using the electrolytic hydrogen gas inhaler 1 (sample use week), so that a crossover trial was conducted between the two groups for two periods. In a week of hydrogen inhalation, hydrogen was inhaled by the subject using the electrolytic hydrogen gas inhaler 1 for five minutes per dose, five times per day. In a week without hydrogen inhalation, hydrogen is not inhaled.

The contents of the test will be described below.

The subject was asked to fill in a typical psychological question sheet about sleep quality evaluations (scores of 0 to 27) and stress evaluations (scores of 0 to 30) at the start of the test (0 W), after a week (1 W), and after two final weeks (2 W).

1)Sleep questionnaire (Pittsburgh Sleep Quality Index in Japanese; PSQI-J)

2)Stress check list, 30 items (SCL30)

Moreover, in a test period, the subject was asked to record a sleeping time, a wake-up time, and body conditions in a log. As a physiological measurement of sleep, the subject in the test period (except for a bath time) was asked to wear a small activity tracker (Micro Tag activity tracker MTN-220) at the center of the belly of the subject and measure an activity during daytime and sleep. As specific measurement data, sleep variables (bedtime, sleep-onset time, waking time, the number of tosses and turns, the number of arousals during sleep, sleep efficiency, and a total sleep time or the like) in three weekdays (Tuesday, Wednesday, Thursday) were determined by a sleep-wake rhythm study program (registered trademark, Sleep Sign Act) and then data on the variables was analyzed.

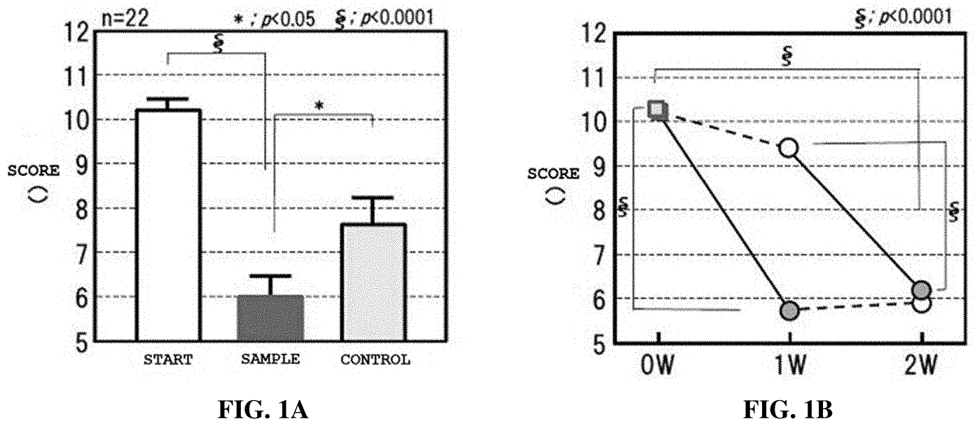

FIGS. 1A and 1B indicate the analysis results of the PSQI-J total scores of the group A and the group B according to the sleep questionnaire (Pittsburgh Sleep Quality Index). FIG. 1A indicates, from the left, a PSQI-J score at the start of the test, a PSQI-J score at night in a hydrogen inhalation week (sample use week), and a PSQI-J score at night in a nonuse week (control week). The PSQI-J score at the start of the test was 10.18±0.36, whereas the PSQI-J score at night in the hydrogen inhalation week significantly decreased to 6.00±0.49. The PSQI-J score at night in the nonuse week was 9.36±0.77, which did not make a difference. This proves that subjective sleep quality significantly improved at night in the hydrogen inhalation week as compared with sleep quality at night in the nonuse week.

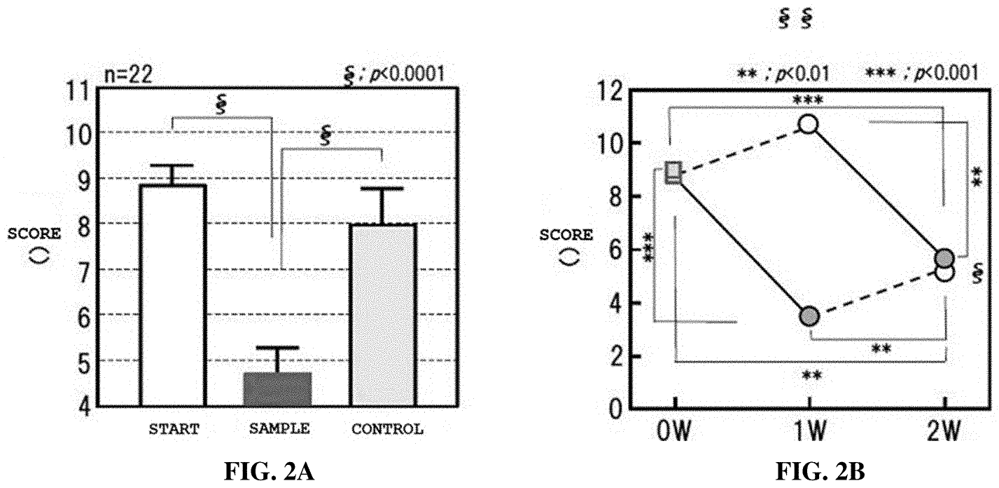

FIGS. 2A and 2B indicate the analysis results of the total scores of the group A and the group B according to the stress check list, 30 items (SCL30). Specifically, as in FIGS. 1A and 1B, changes of stress are analyzed in FIGS. 2A and 2B from an SCL30 score at the start of the test, an SCL30 score at night in a hydrogen inhalation week (sample use week), and an SCL30 score at night in a nonuse week (control week). From the analysis results, the SCL30 score had an initial value of 8.81±0.59 at the start of the test and significantly decreased (p<0.0001) to 4.72±0.59 at night in the hydrogen inhalation week with a large effective dose (r=0.62). The SCL30 score at night in the nonuse week was 8.00±0.89, which did not make a difference.

The effective dose r value is a correlation coefficient indicating the strength and direction of the relationship between hydrogen inhalation and each verification test, r=±1 indicates the strongest correlation, and ±0 indicates no correlation. In this case, according to generalizations, it is determined that r<0.10 indicates a small correlation, 0.10≤r<0.50 indicates a medium correlation, and r≥0.50 indicates a large correlation. Moreover, it is assumed that a p value indicates a significance probability. In other words, if hydrogen inhalation and each test are irrelevant to each other, the p value indicates the probability of the result (contingency). A smaller p value indicates that hydrogen inhalation is related to influence in each verification test.

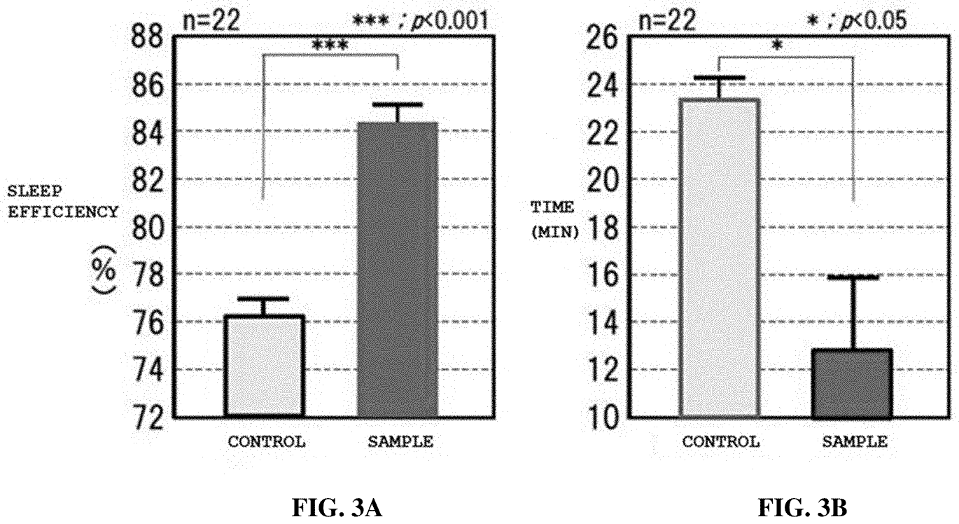

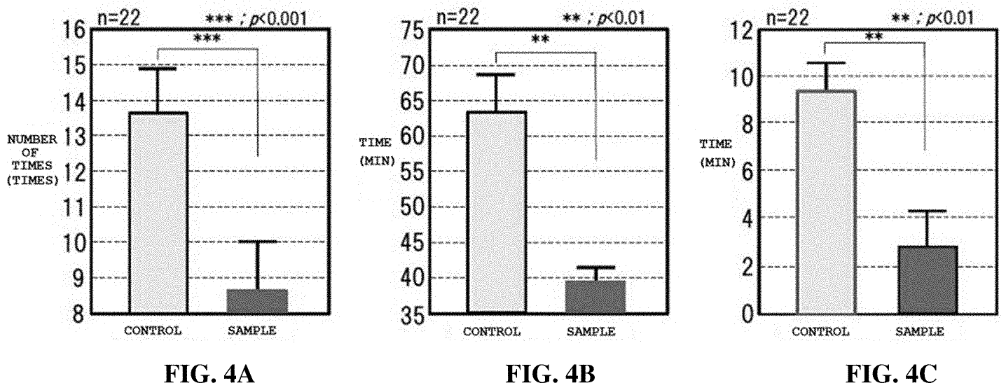

FIGS. 3A-3B and FIGS. 4A-4C indicate the analysis results of physical activities during sleep. Specifically, FIG. 3A indicates the analysis result of sleep efficiency, FIG. 3B indicates the analysis result of a sleep-onset time (sleep latency time), FIG. 4A indicates the analysis result of the number of tosses and turns, FIG. 4B indicates the analysis result of an arousal time during sleep, and FIG. 4C indicates the analysis result of out-of-bed time (out-of-bed latency time). A sleep-onset time (FIG. 3B) was 23 minutes and 49 seconds at night in a nonuse week and was 11 minutes and 49 seconds at night in a hydrogen inhalation week, indicating a significant reduction (p<0.05) with a medium effective dose (r=0.48). Out-of-bed time (FIG. 4C) was 9 minutes and 20 seconds at night in a nonuse week and was 5 minutes and 38 seconds at night in a hydrogen inhalation week, indicating a significant reduction (p<0.01) with a large effective dose (r=0.61). Sleep efficiency (FIG. 3A) was 75.5±2.6% at night in a nonuse week and was 84.2±2.1% at night in a hydrogen inhalation week, indicating a significant increase (p<0.001) with a large effective dose (r=0.69).

Thus, by continuously using hydrogen inhalation, particularly by continuous hydrogen inhalation using at least the electrolytic hydrogen gas inhaler 1 under the foregoing conditions, a significant decrease in stress, an improvement in awareness of sleeplessness, a significant increase in sleep efficiency, and significant reductions in sleep-onset time and out-of-bed time are confirmed as compared with the case where hydrogen inhalation is unused. This proved that the continuous use of hydrogen inhalation reduces daily stress and improves sleep quality.

The amount of hydrogen generated per unit time by the electrolytic hydrogen gas inhaler 1 used in the verification test is 8 cc per minute according to electrolysis (4 cc of oxygen is also generated at the same time), so that 12 cc of mixed gas of oxygen and hydrogen is generated per minute. The mixed gas is inhaled under natural breathing. It is known that an adult normally inhales about five liters of air on average per minute. On the assumption that generated mixed air is fully inhaled, mixed gas contained in expired gas was theoretically calculated to be up to 0.24% (hydrogen: 0.18%, oxygen:

Moreover, gas generated from the electrolytic hydrogen gas inhaler 1 is hydrogen and oxygen, and a hydrogen concentration and an oxygen concentration in mixed gas are both increased relative to the air. As described above, a hydrogen concentration is increased by 0.18% and an oxygen concentration is increased by 0.06%, whereas a hydrogen concentration is 0.5×10⁻⁴% (=0.5 ppm) and an oxygen concentration is about 21% in the air. Thus, it can be assumed that an increase in oxygen concentration in mixed gas is quite small and a contribution is substantially made to an increase in hydrogen concentration.

Furthermore, an examination of the above verification test proves that continuous inhalation is more significant than the total amount of hydrogen inhalation. In the present verification test, hydrogen inhalation was continued for five minutes per dose and was conducted five times per day according to a verification of the setting and effect of a lower threshold value on the basis of a balance between an amount of hydrogen in blood and the probability of continuous use by a user. First, the lower threshold value of hydrogen inhalation is set at five minutes per dose based on a reperfusion phenomenon and the effects of antioxidation and bloodstream restoration. The reperfusion phenomenon is recognized when ischemia is induced in an organ and a bloodstream is occasionally restored. The phenomenon is significant in the restoration of a bloodstream.

Moreover, it is found that the effect of antioxidation and bloodstream restoration by a hydrogen concentration, which is increased in blood by hydrogen inhalation, is considerably lessened when hydrogen inhalation is stopped five minutes before reperfusion, and a certain effect is obtained by inhalation five minutes before reperfusion. In the method for improving sleep according to the present invention, a main object is to support sleep improvement for healthy people not to be treated. Since subjects cannot be restricted by prescriptions, it is significant to provide the lower limit of a useful inhalation time and cycle such that subjects can make a habit of continuous use of hydrogen. From this viewpoint, an inhalation time for each inhalation was set at five minutes as the lower threshold value based on the reperfusion phenomenon and the effects of antioxidation and bloodstream restoration, and the usefulness was proved.

Hydrogen inhalation was conducted five times, which is a lower threshold value, per day. The lower threshold value is set on the basis of a balance between an amount of hydrogen in blood and the probability of continuous use by a user, proving that the effect of sufficiently improving sleep is obtained by five times as the lower threshold value in the present verification test. It is found that hydrogen in blood falls below an effective concentration and disappears in about an hour. In this respect, hydrogen may be inhaled 24 times per day or as many as the number of waking hours (any side effect by the continuous use of hydrogen is not found and thus an upper threshold value is not specified), but in consideration of the main object that is to support sleep improvement for healthy people not to be treated, it is significant to provide the lower limit of a useful inhalation time and cycle such that subjects can make a habit of continuous use of hydrogen. Ordinary supplements, which are not primarily intended for treatment and are close to hydrogen inhalation, are typically taken once a day, for example, every morning or night, after waking up, or before bedtime, or three times a day, for example, before or after meals. Three times a day before and after meals as the most common administration are equivalent to the maximum number of doses in habits. In consideration of the feature of support for sleep improvement, two times after waking up and before bedtime may be incorporated into habits, so that it is reasonable to recommend five times in total per day as the lower threshold value of the number of hydrogen inhalations that support the sleep improvement of healthy people (six or more times are further recommendable). From this viewpoint, an inhalation time for five inhalations per day was set as a lower threshold value in the present verification test, and the effect the inhalation time was proved.

<<Electrolytic Hydrogen Generator>>

An example of an electrolytic hydrogen generator used for the method for supporting sleep improvement according to the present invention will be described below.

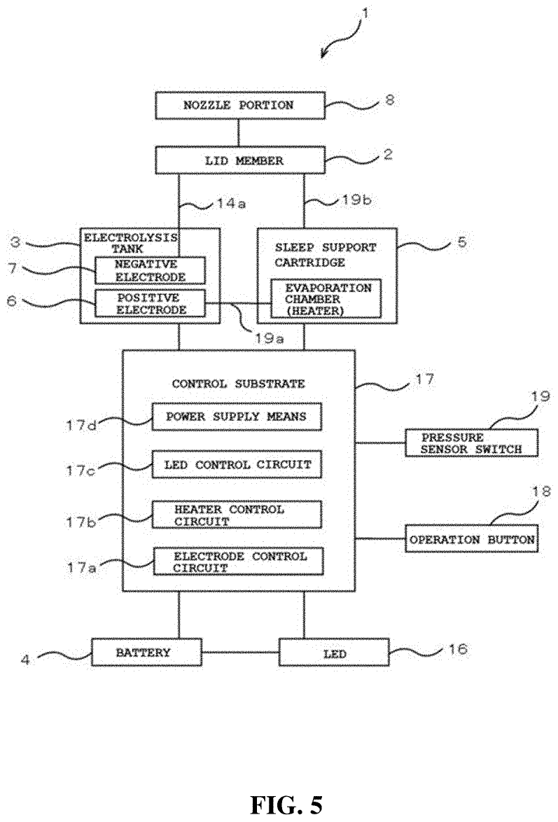

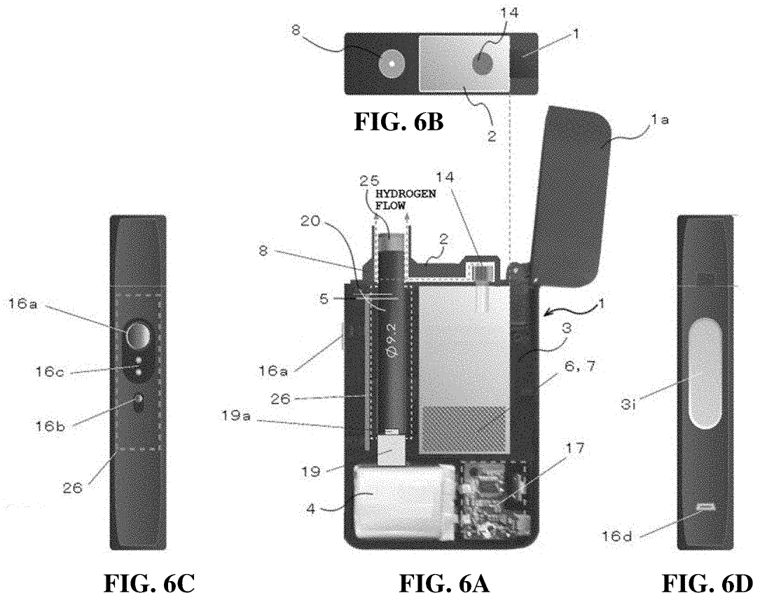

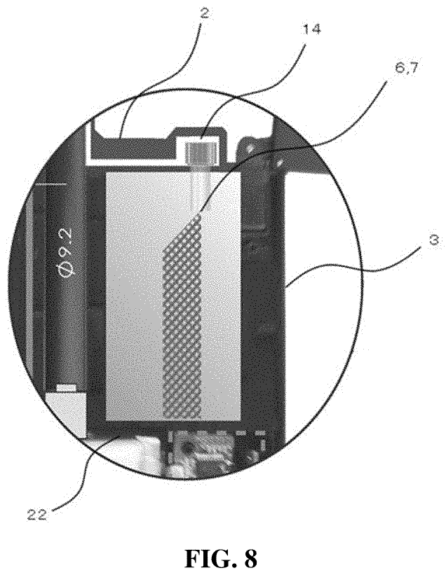

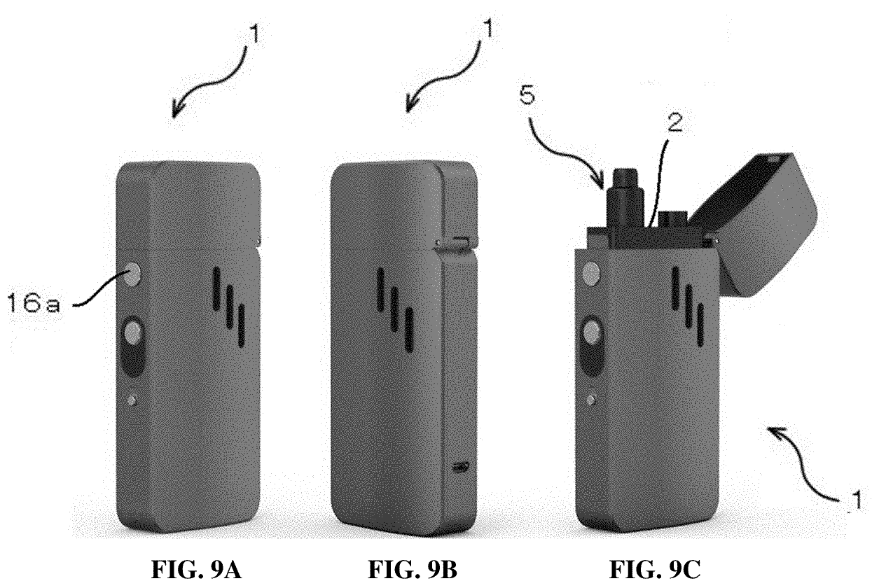

An embodiment of an electrolytic hydrogen generator 1 will be illustrated below. FIG. 5 illustrates a block diagram schematically illustrating the embodiment, FIGS. 6A-6D include six surfaces in a representative example of the embodiment of FIG. 5, and FIG. 8 illustrates a schematic diagram illustrating a state of electrolysis in the electrolysis tank of the electrolytic hydrogen generator. FIGS. 9A-9C schematically illustrate a specific configuration of an electrode. The electrolytic hydrogen generator 1 of the present invention is not limited to the illustration. It is needless to say that the drawings and the contents of a description may be modified within the scope of ordinary common sense.

As illustrated in FIG. 5, the electrolytic hydrogen generator mainly includes a battery 4, an LED 16, control means 17, an electrolysis tank 3, a sleep support cartridge 5, a the lid member 2, and a nozzle portion 8. First, the battery 4 is rechargeable, and the electrolysis tank 3 includes a pair of positive and negative electrodes 6 and 7. The positive and negative electrodes 6 and 7 receive power from the battery 4 via control means 17, and the LED 16 is connected to the battery 4. The control means 17 includes an electrode control circuit 17a, a heater control circuit 17b, an LED control circuit 17c, and power supply means (power supply circuit) 17d.

Furthermore, a pressure sensor switch 19 is provided at the bottom of the receiving portion of the sleep support cartridge 5. When the lower end of the sleep support cartridge presses the pressure sensor switch 19, the power of the battery 4 is supplied to the sleep support cartridge 5 by the power supply means 17d of the control means 17.

When a user operates an operation button 18, the electrode control circuit 17d controls energization/shutdown of the pair of electrodes 6 and 7 in the electrolysis tank 3 in response to the operation, changes the amount of power supplied from the battery 4 by the power supply means 17d, and supplies the power to the electrodes 6 and 7. The power supply to the pair of electrodes 6 and 7 electrolyzes water stored in the electrolysis tank 3, generates oxygen near the positive electrode 6, and generates hydrogen near the negative electrode 7.

Hydrogen generated from the negative electrode 7 flows into the lid member 2 through an attachment 14 on the electrolysis tank 3. Oxygen generated from the positive electrode 6 is vented.

When the pressure sensor switch 19 is turned on, the power supply means 17d supplies power from the battery 4 to a heater in the sleep support cartridge 5, thereby heating a cartridge with aroma components or supplement adsorbed for enhancing a sleeping effect, the cartridge being attached to an internal steam chamber (not illustrated). When the heater heats the cartridge with supplement adsorbed (including drugs) and aroma components adsorbed (hereinafter simply referred to as “supplement”), supplement-containing steam is generated.

The supplement-containing steam generated in the sleep support cartridge 5 is released into a mouth by inhalation through the nozzle portion 8. By a negative pressure generated by the inhalation, hydrogen released from the attachment 14 flows to the nozzle portion 8 into the lid member 2 so as not to touch a heat source such as the heater via a dedicate flow path, passes through a gap between a portion around the top portion of the sleep support cartridge 5, which is exposed in the lid member 2, and the inner wall of the nozzle portion 8, is mixed with supplement-containing air, and is guided into the mouth. A supplement used in the sleep support cartridge 5 more desirably contains components for directly supporting sleep but plays an important role in continuing hydrogen inhalation without tiredness to acquire an amount of inhalation. This means not only components for supporting sleep but also selecting aromas and the like preferred by users to sufficiently enhance the sleeping effect by hydrogen inhalation.

FIGS. 6A-6D illustrate a specific configuration example of the electrolytic hydrogen generator 1. FIG. 6A is a front view of the electrolytic hydrogen generator 1, FIG. 6B is a top view, FIG. 6C is a left side view, and FIG. 6D is a right side view. FIG. 6A illustrates a state in which the lid member 2 of the electrolytic hydrogen generator 1 is detached. The electrolytic hydrogen generator 1 includes a cylindrical cartridge receiving portion (hereinafter also referred to as “receipting portion”) 20 extending downward from an opening on the upper right side while the lid member 2 is detached (opened). The sleep support cartridge 5 is inserted into the receiving portion 20. The sleep support cartridge 5 is a replaceable component of the body part of a commodity electronically heated tobacco that is cylindrically shaped.

If the sleep support cartridge 5 is turned on by a negative pressure generated by inhalation from the top portion of the sleep support cartridge 5 and turns on a main power supply, which will be described later, power is supplied from a rechargeable battery in the battery 4, the steam chamber is heated by the heater, and supplements or aromatic components are released. When power is supplied from the battery 4 while a negative pressure is applied by inhalation through the upper end of the sleep support cartridge 5, the LED 16 on the lower end of the battery 4 illuminates.

Referring to FIGS. 6A-6D again, the electrolytic hydrogen generator 1 will be described below. The sleep support cartridge 5 is inserted into the receiving portion 20 of the electrolytic hydrogen generator 1. The pressure sensor switch 19 is disposed at the bottom of the receiving portion 20, and a convex screw 19a shaped like the attachment 14 is provided as an electric terminal on the upper end of the pressure sensor switch 19. When the pressure sensor switch 19 is pressed, power from the rechargeable battery (lithium battery) 4 is supplied to the convex screw 19a, allowing inhalation of supplement-containing steam.

On the right side (see FIG. 6C) of the electrolytic hydrogen generator 1, a sleep support cartridge ON/OFF switch 16c, an LED indicator 16b, and a main power supply/hydrogen button 16a are provided. The sleep support cartridge ON/OFF switch 16c is an ON/OFF switch of the pressure sensor switch 19. When the sleep support cartridge ON/OFF switch 16c is turned on, an attachment 5a on the lower end of the sleep support cartridge 5 is coupled and pressed to the convex screw 19a so as to supply power from the rechargeable battery 4 to the sleep support cartridge 5. When the sleep support cartridge ON/OFF switch 16c is turned off, power is not supplied from the rechargeable battery 4 in response to a press to the pressure sensor switch 19. The main power supply/hydrogen button 16a is a button power supply switch for the positive and negative electrodes 6 and 7 in the electrolysis tank 3, which will be described later, and the main power supply, and turns on/off the main power supply or turns on/off power supply to the positive and negative electrodes 6 and 7 depending upon a pressing manner/time.

In this example, when the main power supply/hydrogen button 16a is pressed and held for a predetermined time or is continuously pressed, the positive and negative electrodes 6 and 7 are energized for five minutes to generate hydrogen, and the energization is continued until a user keeps pressing the button or after a lapse of a predetermined time (five minutes or longer). The main power supply is automatically turned off after a lapse of the predetermined time or the button is continuously pressed. In the method for supporting sleep improvement, as described above, a hydrogen inhalation time of five minutes or longer per dose is recommended as a lower limit. Hydrogen is supplied with a circuit configuration where the main power supply is automatically turned off 20 minutes after a user stops pressing the button if the electrodes are energized by a continuous press by the user or 20 minutes after the electrodes are de-energized if the electrodes are de-energized after a lapse of a predetermined time (five minutes or longer). When the hydrogen generator is left after hydrogen inhalation is stopped, a time before the main power supply is automatically turned off is set based on sleep-onset time data obtained in the present verification test. An average sleep-onset time was 23 minutes, 49 seconds in the absence of continuous use of hydrogen inhalation (nonuse time), whereas the sleep-onset time decreases to 9 minutes, 20 seconds on average after one week of continuous use of hydrogen inhalation (use time). Thus, in consideration of 23 minutes, 49 seconds at the start of continuous use (nonuse time)−a recommended inhalation time of five minutes=18 minutes, 49 seconds and 5-minute intervals as reasonable intervals of measurement, it is preferable to automatically turn off the main power supply at 20 minutes, 25 minutes, . . . , and the best mode is assumed to be 20 minutes in view of a power-saving effect. Thus, the electrolytic hydrogen generator 1 as a dedicated device of the method for supporting sleep improvement also uses a control configuration in which the main power supply is turned off 20 minutes after hydrogen inhalation is stopped.

Even if an operation for turning off the main power supply is not performed, the main power supply is automatically turned off after a long time (e.g., 20 minutes). Moreover, the main power supply/hydrogen button 16a illuminates during the generation of hydrogen and has the function of indicating the remaining power of the rechargeable battery 4 according to a color of illumination. In this example, the button illuminates blue when the remaining power is 20 to 80%, and the button illuminates white when the remaining power is 80 to 100%. The LED indicator 16b includes two vertically arranged LEDs. The upper LED illuminates when power is supplied to the positive and negative electrodes 6 and 7 in the electrolysis tank 3, and the lower LED illuminates when the pressure sensor switch 19 is turned on to energize the sleep support cartridge 5. The illumination of the the sleep support cartridge ON/OFF switch 16c, the LED indicator 16b, and the main power supply/hydrogen button 16 is controlled by an internal indicator basement 26.

As described above, when the pressure sensor switch 19 is turned on, power from the rechargeable battery 4 is supplied to the pair of positive and negative electrodes 6 and 7 by the control means 17. As illustrated in FIG. 6C, the pair of positive and negative electrodes 6 and 7 may be horizontally disposed at the inner bottom of the electrolysis tank 3 or may be vertically disposed as illustrated in FIG. 5. The rechargeable battery 4 is rechargeable by power from a USB terminal 16d on the side of the electrolytic hydrogen generator 1 (see FIG. 6D).

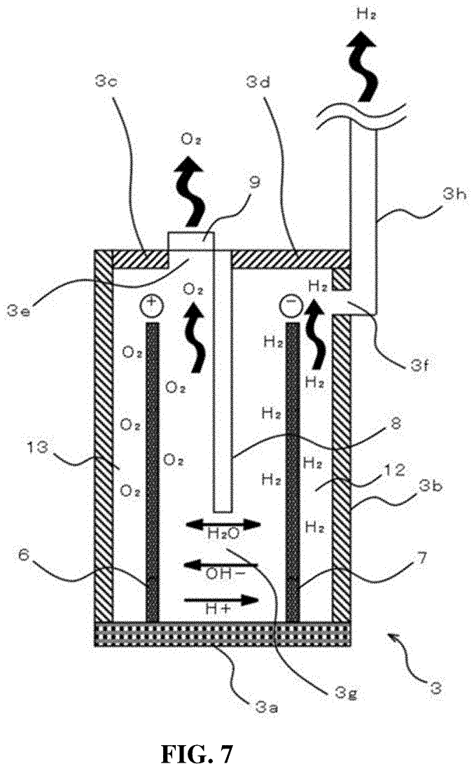

Referring to FIG. 7, a configuration in the electrolysis tank 3 and a state of electrolysis in the electrolysis tank 3 with the energized positive and negative electrodes 6 and 7 will be described below. The electrolysis tank 3 containing water as illustrated in FIG. 7 mainly includes a cylindrical member 3b that is hollow and extends in the longitudinal direction, a bottom member 3a that closes the bottom of the cylindrical member 3b, and lid members 3c and 3d (3c and 3d may be integrated) that close the top portion of the cylindrical member 3a. Energization of the positive and negative electrodes 6 and 7 generates oxygen (O₂) near the positive electrode 6 and generates hydrogen (H₂) near the negative electrode 7. The generated oxygen and hydrogen have a lower specific gravity than water and thus move upward to a gap 3g. In this configuration, the electrolysis tank 3 has a partition member 8 that extends downward from the upper end of the electrolysis tank 3 so as to divide the electrolysis tank 3 into a hydrogen-gas generation layer 12 near the negative electrode 7 and an oxygen-gas generation layer 13 near the positive electrode 6. At the lower end of the partition member 8, the gap 3g is provided from the top surface of the bottom member 3a so as to fluidly connect the hydrogen-gas generation layer 12 and the oxygen-gas generation layer 13.

The partition member 8 interferes with mixing of oxygen and hydrogen in the electrolysis tank 3 when oxygen and hydrogen move upward. Under the gap 3g provided at the lower part of the partition member 8, water (H₂O) is not divided by the partition member 8 and thus can freely move, that is, ions (“OH⁻” and “H⁺”) necessary for generating oxygen and hydrogen can move. In this way, the partition member 8 can interfere with mixing of oxygen and hydrogen while allowing electrolysis.

The lid member 3c closes the top portion of the oxygen-gas generation layer 13 and has an opening 3e between a part of the lid member 3c or the lid member 3c and the partition member 8 or the cylindrical member 3b. The opening 3e is closed by an oxygen permeable membrane 9. Thus, even if hydrogen leaks from the hydrogen-gas generation layer 12 to the oxygen-gas generation layer 13 through the gap 3g or the like, gas released to the outside is limited to oxygen by the oxygen permeable membrane 9. The oxygen permeable membrane 9 may be disposed on an electrolyte solution inlet/hydrogen generation port 14 (described later) in FIGS. 6A-6D but is preferably disposed on a hole specific for the electrolysis tank 3.

Moreover, the hydrogen-gas generation layer 12 has the lid member 3d that closes the top portion of the hydrogen-gas generation layer 12 and an opening 3f provided near the hydrogen-gas generation layer 12 and at the top portion of the cylindrical member 3b. The opening 3f connects to a bypass passage 3h. Thus, hydrogen generated at the negative electrode 7 in hydrogen-gas generation layer 12 passes through the bypass passage 3h and flows upward.

Regarding the passage of hydrogen from the opening 3f to the bypass passage 3h in FIG. 7, in the example of FIGS. 6A-6D, the electrolyte solution inlet/hydrogen generation port 14 corresponds to the opening 3f and a gap between the top portion of the electrolysis tank 3 and the lid member 2 corresponds to the bypass passage 3h. The electrolyte solution inlet/hydrogen generation port 14 has, as described above, the function of an inlet for injecting an electrolyte solution or water into the electrolysis tank 3 and the function of the opening 3f for releasing hydrogen from the electrolysis tank 3 to the outside. The electrolyte solution inlet/hydrogen generation port 14 is shaped to be removable screwed, and an electrolyte solution or water is injected from the opening 3f after the port is unscrewed and detached. In a screwed state, the leakage of an electrolyte solution or the like in the electrolysis tank 3 is suppressed and hydrogen is released from a permeable membrane (not illustrated) of gas such as hydrogen, the permeable membrane being additionally provided to close a hole or the like at the electrolyte solution inlet/hydrogen generation port 14.

As indicated by dotted lines in FIGS. 6A-6D, released hydrogen flows in a dedicated flow path(not shown) to the left (to the sleep support cartridge 5) in the lid member 2. The lid member 2 has a cylindrical nozzle portion 2a on the left end, the nozzle portion 2a protruding with an opening on the upper end. The lid member 2 is an integrated member that is removably disposed at the top portion of the electrolysis tank 3 while covering the upper end of the sleep support cartridge 5 and the electrolyte solution inlet/hydrogen generation port 14. When the lid member 2 is attached to the top portion of the electrolysis tank 3, the upper end of the sleep support cartridge 5 is nested into the opening of the nozzle portion 2a with a clearance 26 formed around the cartridge. By inhalation through the nozzle portion 2a, hydrogen (air containing high-concentration hydrogen) flowing through the dedicated flow path from the electrolyte solution inlet/hydrogen generation port 14 moves upward in the clearance 26 and is released to the outside (see the dotted lines in FIGS. 6A-6D). This allows intake of high-concentration hydrogen. Moreover, power supply to the sleep support cartridge 5 allows intake of mixed gas of high-concentration hydrogen and air containing a supplement and/or aromatic components with an enhanced sleeping effect. The electrolytic hydrogen generator 1 has an open/close cover 1a that is opened in the example of FIGS. 6A-6D. Furthermore, an opening (electrolyte solution check window) 3i for checking a fluid volume in the electrolysis tank 3 is provided on the side of the electrolytic hydrogen generator 1, thereby visually checking a fluid volume in the electrolysis tank 3.

For reference, FIGS. 9A-9C show external view photographs of the embodiment of the electrolytic hydrogen generator 1 according to the present invention. FIG. 9A illustrates the front of the electrolytic hydrogen generator 1 viewed from diagonally left, FIG. 9B illustrates the front of the electrolytic hydrogen generator 1 viewed from diagonally right, and FIG. 9C illustrates the front of the electrolytic hydrogen generator 1 viewed from diagonally left with the opened cover 1a. Although some parts are different in design from the example of FIGS. 6A-6D, main structures are substantially identical to those of FIGS. 6A-6D, and reference numerals in FIGS. 9A-9C are identical to those of the example of FIGS. 6A-6D.

The embodiment was described about the method for supporting sleep improvement for a user who is aware of sleeplessness and a proper electrolytic hydrogen generator used for the method according to the present invention. The present invention is not limited to the embodiment, and a person skilled in the art could understand that other modification examples and improvement examples can be obtained without departing from the scope of claims and the spirit and teachings of a description in the specification.

### REFERENCE SIGNS LIST

1 Electrolytic hydrogen generator

2 Lid member

3 Electrolysis tank

4 Rechargeable battery

5 Sleep support cartridge

6 Positive electrode

7 Negative electrode

8 Nozzle portion

17 Control means

18 Operation button (Operation means)

19 Pressure sensor switch

19a Convex screw

20 Cartridge receiving portion

25 Cartridge with supplement adsorbed

1. A method for supporting sleep improvement of a user who is aware of sleeplessness, in which air containing high-concentration hydrogen is continuously inhaled from a nozzle portion of a portable electrolytic hydrogen generator for a predetermined time or longer each time through a mouth or a nose multiple times per day, by continuously using the electrolytic hydrogen generator that releases air containing high-concentration hydrogen from the nozzle portion when necessary.

2. The method for supporting sleep improvement according to claim 1, wherein air containing high-concentration hydrogen is inhaled from the nozzle portion of the electrolytic hydrogen generator through a mouth or a nose for about five minutes or longer per dose and at least five times per day, and the inhalation is continuously used for about two weeks or longer.

3. A portable electrolytic hydrogen generator used for supporting sleep improvement for a user who is aware of sleeplessness, by continuously using inhalation of air containing high-concentration hydrogen through a mouth or a nose, the electrolytic hydrogen generator comprising:
a body cover member including a battery, a control substrate for controlling power supply from the battery, and a pair of positive and negative electrodes in which a positive electrode and a negative electrode are energized or shut down by the control substrate;
a transparent or translucent electrolysis tank that is attached to the body cover member, accommodates the inserted pair of positive and negative electrodes, and is capable of storing water;
a mixing part including a passage that fluidly connects a nozzle portion and the electrolysis tank via a dedicated path and draws ambient air, the nozzle portion allowing inhalation through a mouth or a nose while being held with one hand; and
operation means enabling an operation of the electrolytic hydrogen generator held with one hand,
wherein the control substrate controls power supply and stop of power supply from the battery to the positive and negative electrodes in response to an operation of the operation means, the control including control for supplying power for a predetermined time in response to an operation of the operation means and stopping power supply after a lapse of the predetermined time.

4. The electrolytic hydrogen generator according to claim 3, wherein the operation means has a single operation button allowing operation by pressing, the control substrate performs control to supply power from the battery to the positive and negative electrodes when the operation button is pressed and a pressing state is kept while a main power supply is turned on, and the control substrate performs control to stop power supply from the battery to the positive and negative electrodes when the pressing state is released.

5. The electrolytic hydrogen generator according to claim 3, wherein the operation means has a single operation button allowing operation by pressing, the control substrate performs control to supply power from the battery to the positive and negative electrodes when the operation button is pressed once while a main power supply is turned on, and the control substrate performs control to stop power supply from the battery to the positive and negative electrodes after a lapse of a preset time.

6. The electrolytic hydrogen generator according to claim 4, wherein the operation means performs control to turn off the main power supply after a lapse of a predetermined time following the stop of power supply from the battery to the positive and negative electrodes.

7. The electrolytic hydrogen generator according to claim 3, further comprising an LED that illuminates the electrolysis tank, and
the control substrate energizes the LED when power is supplied from the battery to the positive and negative electrodes.

8. The electrolytic hydrogen generator according to claim 3, comprising:
a sleep support cartridge that is heated by energization and releases gas containing aromatic components or a supplement with an enhanced sleeping effect,
wherein the control means controls energization or shutdown to the pair of electrodes and/or the sleep support cartridge, and
hydrogen and gas containing aromatic components or a supplement released by energizing the sleep support cartridge are mixed in the mixing part and are guided to the nozzle portion for inhalation through a mouth or a nose, the hydrogen being generated by energizing the pair of positive and negative electrodes so as to electrolyze an electrolyte solution or water in the electrolysis tank.

9. The electrolytic hydrogen generator according to claim 5, wherein the operation means performs control to turn off the main power supply after a lapse of a predetermined time following the stop of power supply from the battery to the positive and negative electrodes.

10. The electrolytic hydrogen generator according to claim 4, further comprising an LED that illuminates the electrolysis tank, and
the control substrate energizes the LED when power is supplied from the battery to the positive and negative electrodes.

11. The electrolytic hydrogen generator according to claim 5, further comprising an LED that illuminates the electrolysis tank, and
the control substrate energizes the LED when power is supplied from the battery to the positive and negative electrodes.

12. The electrolytic hydrogen generator according to claims 4, comprising:
a sleep support cartridge that is heated by energization and releases gas containing aromatic components or a supplement with an enhanced sleeping effect,
wherein the control means controls energization or shutdown to the pair of electrodes and/or the sleep support cartridge, and
hydrogen and gas containing aromatic components or a supplement released by energizing the sleep support cartridge are mixed in the mixing part and are guided to the nozzle portion for inhalation through a mouth or a nose, the hydrogen being generated by energizing the pair of positive and negative electrodes so as to electrolyze an electrolyte solution or water in the electrolysis tank.

13. The electrolytic hydrogen generator according to claim 5, comprising:
a sleep support cartridge that is heated by energization and releases gas containing aromatic components or a supplement with an enhanced sleeping effect,
wherein the control means controls energization or shutdown to the pair of electrodes and/or the sleep support cartridge, and
hydrogen and gas containing aromatic components or a supplement released by energizing the sleep support cartridge are mixed in the mixing part and are guided to the nozzle portion for inhalation through a mouth or a nose, the hydrogen being generated by energizing the pair of positive and negative electrodes so as to electrolyze an electrolyte solution or water in the electrolysis tank.

14. The electrolytic hydrogen generator according to claim 6, comprising:
a sleep support cartridge that is heated by energization and releases gas containing aromatic components or a supplement with an enhanced sleeping effect,
wherein the control means controls energization or shutdown to the pair of electrodes and/or the sleep support cartridge, and
hydrogen and gas containing aromatic components or a supplement released by energizing the sleep support cartridge are mixed in the mixing part and are guided to the nozzle portion for inhalation through a mouth or a nose, the hydrogen being generated by energizing the pair of positive and negative electrodes so as to electrolyze an electrolyte solution or water in the electrolysis tank.

15. The electrolytic hydrogen generator according to claim 7, comprising:
a sleep support cartridge that is heated by energization and releases gas containing aromatic components or a supplement with an enhanced sleeping effect,
wherein the control means controls energization or shutdown to the pair of electrodes and/or the sleep support cartridge, and
hydrogen and gas containing aromatic components or a supplement released by energizing the sleep support cartridge are mixed in the mixing part and are guided to the nozzle portion for inhalation through a mouth or a nose, the hydrogen being generated by energizing the pair of positive and negative electrodes so as to electrolyze an electrolyte solution or water in the electrolysis tank.
